(19) United States
(12) Patent Application Publication
(10) Pub. No.: US 2024/0024567 A1
(43) Pub. Date: Jan. 25, 2024

(54) SYRINGE PUMP

(71) Applicant: B. Braun Melsungen AG, Melsungen (DE)

(72) Inventor: Stefan Bartsch, Neuenstein (DE)

(21) Appl. No.: 18/353,838

(22) Filed: Jul. 17, 2023

(30) Foreign Application Priority Data
Jul. 19, 2022 (DE) 10 2022 117 992.3

(51) Int. Cl. A61M 5/142; A61M 5/315

(52) U.S. Cl. CPC A61M 5/142; A61M 5/31576; A61M 2205/0294

(57) ABSTRACT

A syringe pump includes a syringe holder for holding a syringe cylinder of an inserted syringe, a drive unit for axial displacement of a piston rod of the syringe, and a brake element which can be moved transversely to the longitudinal direction of the syringe via a brake actuator. The brake element is movable from a release position to a brake position and back. The brake element contacts the piston rod in the brake position. A dosing accuracy of the syringe pump is improved by the brake actuator having a piezo element for moving the brake element.

CROSS-REFERENCE TO RELATED APPLICATION(S)

This application claims priority under 35 U.S.C. § 119 to German Application No. 10 2022 117 992.3, filed on Jul. 19, 2022, the content of which is incorporated by reference herein in its entirety.

### FIELD

The present disclosure relates to a syringe pump syringe pump with a syringe holder for holding a syringe cylinder of an inserted syringe, a drive unit for axial displacement of a piston rod of the syringe, and a brake element movable transversely to the longitudinal direction of the syringe via a brake actuator from a release position to a brake position and back, wherein the brake element contacts the piston rod in the brake position.

### BACKGROUND

In the case of syringe pumps of the aforementioned design, the problem arises that the known brake actuators have a relatively long reaction time. After transmission of a brake signal to the brake actuator, it takes this reaction time until the brake element contacts the piston rod and thus fixes it in the axial direction. The reaction time thus has a negative effect on the dosing accuracy of the syringe pump.

When the syringe pump is started up, the drive unit moves onto the piston rod. The piston rod is held via the brake element and axial displacement of the piston rod is prevented. This prevents the unintentional release of liquid when the drive unit moves onto the piston rod during startup.

Such a syringe pump is known from EP 1 329 232 1. The brake actuator disclosed there has a motor arranged on a slide. This motor drives a gear wheel with helical teeth mounted on the slide via a worm wheel. The gear wheel is provided with a threaded bore which engages with a thread of a spindle rod. Turning the gear wheel axially displaces the spindle rod, which is secured against rotation. The spindle rod thus forms a linear actuator, on which the brake element itself is also arranged. The brake actuator thus has a complex mechanical system, which means that a relatively high reaction time is to be expected. Furthermore, temperature differences or wear can have a negative effect on the reaction time, so that the reaction time is not only relatively high, but also the exact amount is not known, which furthermore has a negative effect on the dosing accuracy.

### SUMMARY

The present disclosure is based on the object of providing a syringe pump which reduces or eliminates the problems of the prior art. In particular, the dosing accuracy of the syringe pump is to be improved.

More precisely, the object is solved in that the brake actuator has or is a piezo element for moving the brake element. Such a piezo element has a particularly short reaction time, so that in particular the dosing accuracy of the syringe pump is also improved. After transmission of a brake signal to the piezo element the brake element is quickly moved into its brake position via the piezo element as the brake actuator, so that the piston rod is then fixed in its axial position and no further liquid can be pumped out of the syringe cylinder via the piston rod.

In a preferred embodiment of the syringe pump, the piezo element has a stack of multiple piezo plates electrically connected in parallel.

By forming the piezo element as a stack with multiple piezo plates electrically connected in parallel, the change in length of the piezo element can be increased, which the piezo element can perform when an electrical voltage is applied to the piezo element.

Preferably, the brake element has a knife, blade or pin for axially locking the piston rod in the brake position of the brake element.

Via the knife/blade or the pin, the risk of the piston rod slipping on the brake element is minimized.

Advantageously, the piezo element is connected to a control device and/or regulation device, preferably arranged in the syringe holder, for control and/or regulation of the piezo element.

The control device and/or regulation device is used to meter the liquid pumped from the syringe cylinder via the syringe pump. For this purpose, the control device and/or regulation device is preferably also connected to the drive unit in terms of control technology. A drive signal can be transmitted to the drive unit and a brake signal to the brake actuator via the control device and/or regulation device. Furthermore, the control device and/or regulation device could be data-connected to an input unit, so that via such an input unit the amount of liquid to be dosed by means of the syringe can be transmitted to the control device and/or regulation device of the syringe pump.

It is further preferred to determine the size of the syringe by means of the pressure transmitted from the brake element to the piezo element, via the resulting pressure measurement signal transmitted from the piezo element to the control device and/or regulation device, and via software executable by means of the control device and/or regulation device.

This determination of the size of the syringe represents a possibility to avoid dosing errors due to the assumption of an incorrect syringe size.

In another embodiment of the syringe pump, the syringe cylinder can be supported on one side, over a circumferential range of less than 180° of the syringe cylinder in the radial direction via the syringe holder. The syringe holder is arranged on a side of the syringe cylinder opposite the brake element, and a brake piston rod connects the syringe holder and the brake element to one another.

This ensures that the syringe is held securely in position even when a braking force is applied by the brake element to the piston rod of the syringe. Furthermore, the syringe can be quickly and safely inserted into the desired position for the syringe pump. The brake piston rod is rigid or at least partially movable.

The brake actuator is advantageously arranged on the same side of the syringe cylinder as the brake element. The brake actuator preferably contacts the brake element. This allows the reaction time to be shortened further. Alternatively, it is conceivable to use a transmission (power train) between the brake actuator and the brake element.

Preferably, the brake actuator is connected to the control device and/or regulation device via a cable running through the brake piston rod.

Control and/or regulation signals can be transmitted via the cable. It is also conceivable to supply power to the brake actuator via the cable. Such a power supply is also feasible, for example, with the control device and/or regulation device.

According to another, alternative embodiment of the syringe pump, the brake actuator is arranged on the same side of the syringe cylinder as the syringe holder. Then, via the brake actuator, the brake piston rod can be moved transversely to the longitudinal direction of the syringe to move the brake element from the release position to the brake position and back.

This means that the area of the syringe pump associated with the brake element, which is arranged on the side of the syringe cylinder opposite the syringe holder, can be made particularly small. Furthermore, electrical assembly of the syringe pump can be simplified because all components to be supplied electrically can be arranged particularly close together.

In summary, such a syringe pump is suitable for a wide range of applications, such as for dosing medications present in liquid form via syringes, e.g. into the bloodstream or other body areas of persons.

### BRIEF DESCRIPTION OF THE DRAWINGS

In the following, preferred embodiments are illustrated in more detail with reference to the accompanying figures.

FIG. 1 shows a schematic diagram of a first configuration example of the syringe pump in a side view.

FIG. 2 shows a schematic representation of a section of the first configuration example of the syringe pump in a three-dimensional view.

FIG. 3 shows a schematic diagram of a second configuration example of the syringe pump in a side view.

15 FIG. 4 shows a schematic representation of a section of the second configuration example of the syringe pump in a three-dimensional view.

### DETAILED DESCRIPTION

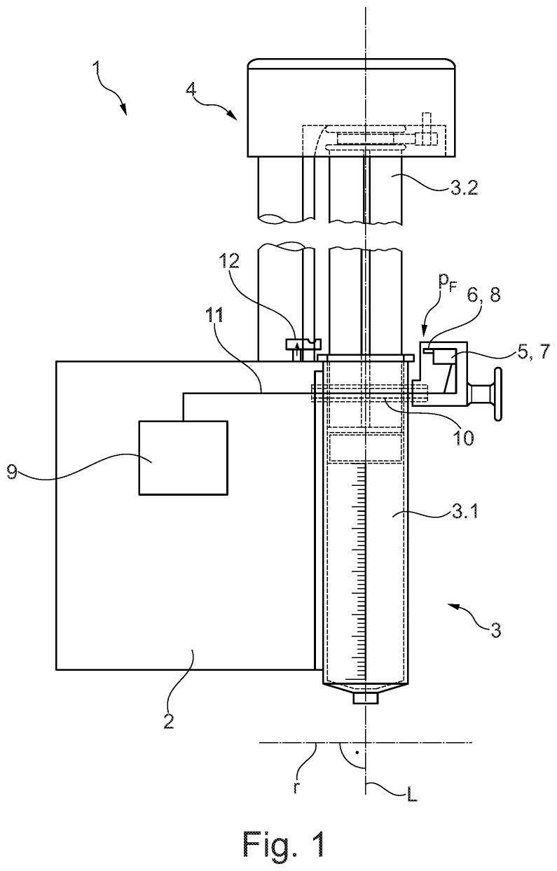

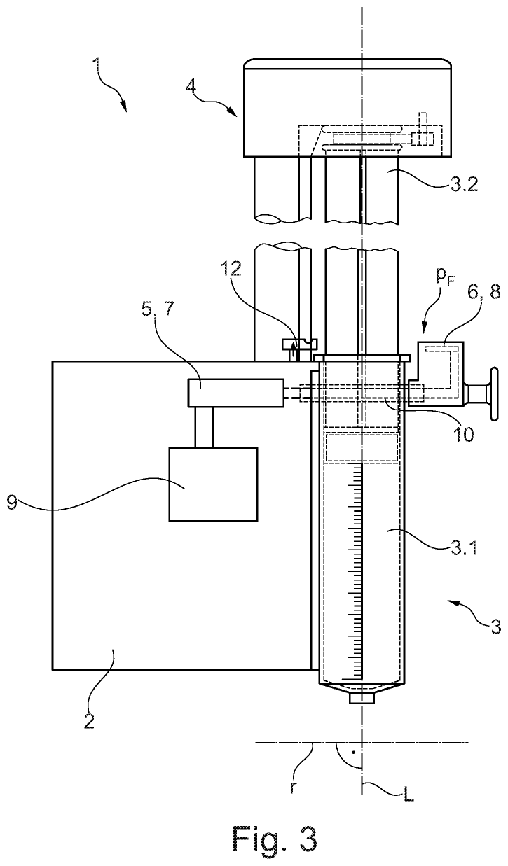

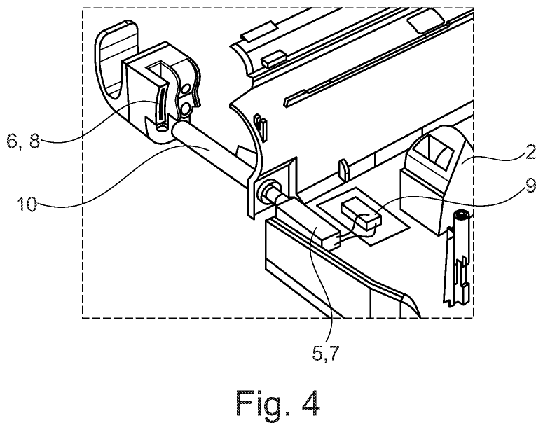

According to FIG. 1 and FIG. 3, the syringe pump 1 has, among other things, a syringe holder 2 for holding a syringe cylinder 3.1 of an inserted syringe 3, a drive unit 4 for axial displacement of a piston rod 3.2 of the syringe 3, and a brake element 6 which can be moved transversely to the longitudinal direction L of the syringe 3 via a brake actuator 5 from a release position p F into a brake position p B and back, and which contacts the piston rod 3.2 in the brake position N.

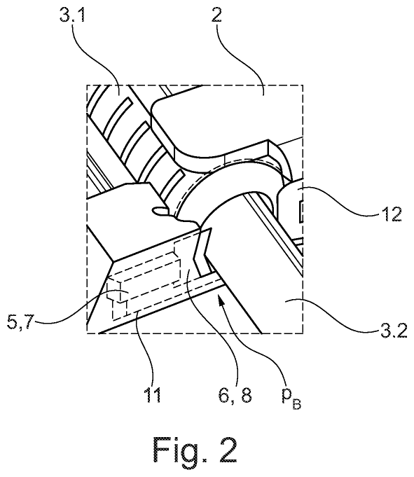

FIG. 1 and FIG. 3 show the brake element 6 in the release position p F . In the three- dimensional view of the first configuration example of the syringe pump 1 from FIG. 2, the brake element 6 is shown in the brake position p B with contact to the piston rod 3.2. FIG. 4 shows the interior of the syringe holder 2 of the second configuration example of the syringe pump 1, wherein the syringe 3 is not shown in FIG. 4 for clarity. The same reference signs are used for identical components in all figures.

The brake actuator 5 has a piezo element 7 for moving the brake element 6.

To actuate the brake actuator 5, a voltage is applied to the piezo element 7. For this purpose, two wires are connected to the piezo element 7 via corresponding lines, as symbolized in the figures. Depending on the polarity of the voltage, the piezo element 7 expands in its axial direction or contracts in its axial direction. The axial direction of the piezo element 7 is the direction in which the change in length of the piezo element 7 leads to the movement of the brake element 6 due to the coupling of the piezo element 7 with the brake element 6.

The piezo element 7 has a stack of multiple piezo plates electrically connected in parallel to achieve the required length change at reasonable voltage values.

The brake element 6 has a knife 8 or a pin for axial locking of the piston rod 3.2 in the brake position p_{B} of the brake element 6.

For its control and/or regulation, the piezo element 7 is connected to a control device and/or regulation device 9 preferably arranged in the syringe holder 2. The control device and/or regulation device 9 is arranged on a printed circuit board as shown in FIG. 4 and can be supplied with power, for example, via a battery that can also be arranged in the syringe holder 2. Alternatively, the syringe pump 1 could be connected to an external power supply via a power cable.

A size of the syringe 3 can be determined with the aid of the pressure transmitted from the brake element 6 to the piezo element 7, the resulting pressure measurement signal transmitted from the piezo element 7 to the control device and/or regulation device 9, and via software executable via the control device and/or regulation device 9. In particular, a diameter of the syringe cylinder 3.1 can be determined. Preferably, a table is available in the control device and/or regulation device 9 30 for determining the size of the syringe 3, in which different pressure measurement values are assigned to different syringe sizes. Such a table is determined, for example, before the syringe pump 1 is used via this or an identically constructed syringe pump and corresponding syringes 3 and stored in the control device and/or regulation device 9. Alternatively, a corresponding functional, modeled dependence of syringe size on pressure measurement signal could also be stored in the control device and/or regulation device 9.

The syringe cylinder 3.1 can be supported on one side, over a circumferential area of less than 180° of the syringe cylinder 3.1 in the radial direction r, via the syringe holder 2. The syringe holder 2 is arranged on a side of the syringe cylinder 3.1 opposite the brake element 6. A brake piston rod 10 connects the syringe holder 2 and the brake element 6 to each other.

According to the first configuration example of the syringe pump 1 from FIG. 1 and FIG. 2, the brake actuator 5 is arranged on the same side of the syringe cylinder 3.1 as the brake element 6. Preferably, the brake actuator 5 contacts the brake element 6, e.g. the knife 8 or the pin is arranged directly on the piezo element 7 and connected to the piezo element 7. The axial direction of the piezo element 7 then runs parallel to the direction of movement of the brake element 6. Preferably, the direction of movement of the brake element 6 and the axial direction of the piston rod 3.2 are essentially perpendicular to each other.

According to FIG. 1 and FIG. 2, the brake actuator 5 is then connected to the control device and/or regulation device 9 via a cable 11 running through the brake piston rod 10.

The cable 11 is preferably two-core. According to FIG. 1 and FIG. 2, the polarity of the voltage applied to the piezo element 7 is preferably selected so that the piezo element 7 expands when voltage is applied and the brake element 6 is then moved from its release position p_{F} to the brake position p B or is subsequently held in the brake position p B , respectively. Alternatively, it is also conceivable that the polarity is selected so that the brake element 6 is in the brake position p_{B} when no voltage is applied to the piezo element 7. When a voltage is applied to the piezo element 7, the brake element 6 is then guided from the brake position p B to the release position p_{F} or, respectively, held in the release position p F by axial contraction of the piezo element 7. The polarity is preferably selected so that the piezo element 7 does not have to be supplied with current for a longer period of time than it has to be supplied with current during operation of the syringe pump 1.

According to the second configuration example of the syringe pump shown in FIG. 3 and FIG. 4, the brake actuator 5 is arranged on the same side of the syringe cylinder 3.1 as the syringe holder 2. Via the brake actuator 5, the brake piston rod 10 can be moved transversely to the longitudinal direction L of the syringe 3 to move the brake element 6 from the release position p F to the brake position p B and back.

In particular, the brake piston rod 10 then has a rigid wooden cylindrical shell and an axially movable, preferably cylindrical core, wherein the core is then connected to the brake actuator 5, in particular to the piezo element 7.

According to FIG. 3 and FIG. 4, the polarity of the voltage applied to the piezo element 7 is preferably selected so that the piezo element 7 contracts when voltage is applied and the brake element 6 is then moved from its release position p F to the brake position p B or is subsequently held in the brake position p B , respectively. Alternatively, it is also conceivable that the polarity is selected so that the brake element 6 is in the brake position p B when no voltage is applied to the piezo element 7. When a voltage is applied to the piezo element 7, the brake element 6 is then guided from the brake position p B to the release position p F or, respectively, held in the release position p F by axial expansion of the piezo element 7. In this second configuration example of the syringe pump 1, the polarity is also preferably selected in such a way that, during operation of the syringe pump 1, the piezo element 7 does not have to be supplied with current for a longer period of time than it has to be supplied with current.

The syringe pump 1 preferably has a clamping mechanism 12 via which the syringe cylinder 3.1 can be fixed after insertion of the syringe 3 into the syringe pump 1. In particular, a syringe wing formed on the syringe cylinder 3.1 can be clamped via the clamping mechanism 12. To remove the syringe 3 from the syringe pump 1, the clamping mechanism 12 is first released.

1. A syringe pump comprising:
a syringe holder for holding a syringe cylinder of an inserted syringe;
a drive unit for axial displacement of a piston rod of the syringe; and
a brake element,
the brake element being moveable transversely to a longitudinal direction of the syringe via a brake actuator from a release position into a brake position and back,
the brake element contacting the piston rod in the brake position, and
the brake actuator comprising a piezo element for moving the brake element.

2. The syringe pump according to claim 1, wherein the piezo element comprises a stack of piezo plates electrically connected in parallel.

3. The syringe pump according to claim 1, wherein the brake element comprises a knife or a pin for axial locking of the piston rod in the brake position of the brake element.

4. The syringe pump according to claim 1, wherein the piezo element is connected to a control device and/or regulation device.

5. The syringe pump according to claim 4, wherein a size of the syringe is determinable by a pressure transmitted from the brake element to the piezo element, by a pressure measurement signal transmitted from the piezo element to the control device and/or regulation device, and by software executable via the control device and/or regulation device.

6. The syringe pump according to claim 1, wherein:
the syringe cylinder is supportable on one side over a circumferential region of less than 180° of the syringe cylinder in a radial direction via the syringe holder,
the syringe holder is arranged on a first side of the syringe cylinder,
the brake element is arranged on a second side of the syringe cylinder opposite the first side, and
a brake piston rod connects the syringe holder and the brake element to one another.

7. The syringe pump according to claim 6, wherein the brake actuator is arranged on the second side of the syringe cylinder.

8. The syringe pump according to claim 7, wherein the brake actuator is connected to the control device and/or regulation device via a cable running through the brake piston rod.

9. The syringe pump according to claim 6, wherein the brake actuator is arranged on the first side of the syringe cylinder, and the brake piston rod is movable transversely to the longitudinal direction of the syringe via the brake actuator in order to move the brake element from the release position to the brake position and back.
